(19) United States
(12) Patent Application Publication
(10) Pub. No.: US 2026/0000808 A1
(43) Pub. Date: Jan. 1, 2026

(54) RADIOPAQUE INJECTABLE SHEAR-THINNING COMPOSITIONS

(71) Applicant: Boston Scientific Scimed, Inc., Maple Grove, MN (US)

(72) Inventors: Boxin Tang, St. Paul, MN (US); Nicolas Ball-Jones, Minneapolis, MN (US); Kent David Harrison, Maple Grove, MN (US); Michael Badger, Princeton, MN (US)

(73) Assignee: Boston Scientific Scimed, Inc., Maple Grove, MN (US)

(21) Appl. No.: 19/252,740

(22) Filed: Jun. 27, 2025

Related U.S. Application Data
(60) Provisional application No. 63/666,323, filed on Jul. 1, 2024.

(51) Int. Cl. A61L 24/04; A61L 24/00; A61L 24/02

(52) U.S. Cl. CPC A61L 24/043; A61L 24/001; A61L 24/02

(57) ABSTRACT

In various aspects, the present disclosure provides injectable shear-thinning compositions that comprise (a) one or more types of fibrous proteins, (b) one or more types of silicate microparticles, (c) one or more types of radiopaque additives, (d) one or more types of block copolymers, and (e) water. Other aspects of the present disclosure pertain to kits that contain such compositions and medical procedures that comprise administering such compositions to a subject.

### CROSS-REFERENCE TO RELATED APPLICATIONS

This application claims the benefit of U.S. Provisional Patent Application Ser. No. 63/666,323 filed on Jul. 1, 2024, the disclosure of which is incorporated herein by reference.

### FIELD

The present disclosure relates to injectable shear-thinning compositions which are radiopaque and can thus be viewed by radiation-based imaging techniques, including X-ray-based imaging techniques. The injectable shear-thinning compositions are useful, for example, in various medical procedures.

### BACKGROUND

Injectable shear-thinning compositions are attractive due to the minimally invasive delivery procedures associated with them, which provide reduced healing time, reduced scarring, decreased risk of infection, and ease of delivery compared with surgically implanted materials. Injectable shear-thinning compositions are especially useful for applications where the final form and shape of the implanted material are either not important or are defined by the void or space into which they are injected. Due to their ease of delivery, injectable shear-thinning compositions are potentially useful in a number of areas, including providing a structural or space-filling function, functioning as embolic agents for diverting or eliminating flow in blood vessels, acting as tissue engineering compositions, and delivery of drugs.

One currently available injectable shear-thinning composition is Obsidio™ Conformable Embolic from Boston Scientific Corporation, Marlborough, Massachusetts, USA. It is pre-packaged in a ready-to-use syringe. As the material is pushed through a catheter on its way to a targeted site during administration, the material shear-thins and flows readily, like a liquid. When shear forces are removed as the material reaches its intended location, it reverts to a soft solid that molds to the targeted vasculature's shape, creating a physical barrier that stops blood flow. The Obsidio™ embolic composition contains laponite, gelatin, water and tantalum and is the only conformable embolic indicated to control bleeding and stop blood flow to tumors in the peripheral vasculature. The Obsidio™ material is radiopaque due to the presence of tantalum particles in its formulation.

### SUMMARY

In various aspects, the present disclosure provides injectable shear-thinning compositions that comprise (a) one or more types of fibrous proteins, (b) one or more types of silicate microparticles, (c) one or more types of radiopaque additives, (d) one or more types of block copolymers, and (e) water.

In some embodiments, the one or more types of fibrous proteins are collagen-based proteins. In some of these embodiments, the types of fibrous proteins comprise gelatin.

In some embodiments, which can be used in conjunction with the above aspects and embodiments, the one or more types of silicate microparticles comprise natural and/or synthetic silicate layered clays.

In some embodiments, which can be used in conjunction with the above aspects and embodiments, the one or more types of radiopaque additives comprise radiopaque microparticles that contain radiopaque metals or radiopaque metal compounds. In some of these embodiments, the radiopaque microparticles are tantalum microparticles.

In some embodiments, which can be used in conjunction with the above aspects and embodiments, the one or more types of block copolymers comprise a polyalkylene oxide block copolymer. In some of these embodiments, the polyalkylene oxide block copolymer comprises one or more polyethylene oxide blocks and one or more polypropylene oxide blocks. In some of these embodiments, the polyalkylene oxide block copolymer is a triblock copolymer that comprises two polyethylene oxide blocks and a central polypropylene oxide block between the polyethylene oxide blocks. In some of these embodiments, the one or more polyethylene oxide blocks have a length between 10 and 500 monomer units and one or more polypropylene oxide blocks have a length between 10 and 300 monomer units.

In some embodiments, which can be used in conjunction with the above aspects and embodiments, the injectable shear-thinning composition further comprises one or more additional agents selected from therapeutic agents, imaging agents, colorants, tonicity adjusting agents, suspension agents, wetting agents, and pH adjusting agents.

In some embodiments, which can be used in conjunction with the above aspects and embodiments, the injectable shear-thinning composition is a sterile composition.

In some embodiments, which can be used in conjunction with the above aspects and embodiments, the injectable shear-thinning composition is provided in a preloaded syringe.

In other aspects, the present disclosure provides kits that comprise a delivery device and one or more containers that contain an injectable shear-thinning composition in any of the above aspects and embodiments.

In some embodiments, the delivery device comprises a syringe, a needle and optionally, a catheter.

In other aspects, the present disclosure provides medical procedures that comprise administering to a subject an injectable shear-thinning composition in accordance with any of the above aspects and embodiments.

In some embodiments, the method comprises injecting the injectable shear-thinning composition into the subject.

In some embodiments, which can be used in conjunction with the above aspects and embodiments, wherein the administering comprises parenteral administration.

In some embodiments, which can be used in conjunction with the above aspects and embodiments, the administering is performed using a catheter or a syringe.

In some embodiments, which can be used in conjunction with the above aspects and embodiments, the administering is performed under image guidance.

Potential benefits of the present disclosure include improved radiopaque agent dispersion in the compositions (thereby increasing radiopacity), improved distal penetration upon injection, and improved composition cohesivity upon injection.

These and other aspects, embodiments and potential benefits of the present disclosure will become immediately apparent to those of ordinary skill in the art upon review of the detailed description and claims to follow.

### BRIEF DESCRIPTION OF THE DRAWINGS

FIG. 1 schematically illustrates a catheter and a syringe that is loaded with an injectable shear-thinning composition, in accordance with an embodiment of the present disclosure.

FIG. 2 illustrates particle size distributions for 20 wt % tantalum particle dispersions in water alone, in water with 0.1 wt % Pluronic® F127, and in water with 0.2 wt % Pluronic® F127, in accordance with embodiments of the present disclosure.

FIG. 3 is an illustration of an arteriovenous malformations (AVM) model for testing injectable shear-thinning formulations in accordance with the present disclosure.

FIG. 4 shows the degree which the AVM model of FIG. 3 is filled upon catheter injection of four injectable shear-thinning formulations at various saline flow rates through the AVM model.

FIG. 5 shows calculated copper thickness (mm) for shear-thinning formulations in accordance with the present disclosure.

FIGS. 6A-6D show plots of voxel count vs. Gray value for shear-thinning formulations in accordance with the present disclosure.

### DETAILED DESCRIPTION

In some aspects, the present disclosure pertains to injectable shear-thinning formulations that comprise (a) a fibrous protein, (b) silicate particles, (c) radiopaque particles, (d) a block copolymer surfactant and (e) water.

A composition is a shear-thinning composition if the viscosity decreases with increasing shear.

In various embodiments, the injectable shear-thinning compositions of the present disclosure are physically crosslinked hydrogel compositions. In some of these embodiments, the injectable shear-thinning compositions of the present disclosure are physically crosslinked by ionic interactions. As used herein, a “hydrogel” refers to a hydrated crosslinked polymer-containing network that does not dissolve when placed in saline.

In some embodiments, the injectable shear-thinning compositions of the present disclosure flow upon application of a pressure greater than a yield stress of the injectable shear-thinning compositions.

Having shear-thinning properties enables the hydrogels of the present disclosure to be injected as a low viscosity, flowing material. Once injection shear is removed, restoration of pre-shear hydrogel rigidity allows the hydrogel to remain localized at the point of injection. When not under shear the hydrogels of the present disclosure are generally in the form of soft solids.

In some embodiments, the injectable shear-thinning compositions of the present disclosure have a pH between 6 and 11, more typically between 8 and 10.

Fibrous proteins are made up of polypeptide chains that are elongated and fibrous in nature or have a sheet-like structure. Fibrous proteins for use in the injectable shear-thinning compositions of the present disclosure include animal-derived proteins and non-animal-derived proteins. Fibrous proteins for use in the injectable shear-thinning compositions of the present disclosure include keratin, elastin, fibroin, myosin, desmin, fibrin, actin, and collagen, including denatured and hydrolyzed forms thereof such as gelatin, polyarginine, polylysine, and spider silk proteins. Specific fibrous proteins for use herein include bovine collagen, porcine collagen, equine collagen, porcine gelatin (e.g., type-A porcine gelatin, gelatin derived from porcine skin, gelatin derived from porcine bones, and the like), bovine gelatin (e.g., type-B bovine gelatin, gelatin derived from bovine skin, gelatin derived from bovine bones, and the like), equine gelatin, avian-derived gelatin and fish-derived gelatin.

In some embodiments, the injectable shear-thinning compositions of the present disclosure contain between 0.1 wt % and 5 wt % or more of one or more types of fibrous proteins, and typically contain between 0.2 wt % and 1 wt % (for example, ranging anywhere from 0.2 wt % to 0.3 wt % to 0.4 wt % to 0.5 wt % to 0.6 wt % to 0.7 wt % to 0.8 wt % to 0.9 wt % to 1 wt %) of or more of one or more types of fibrous proteins, more particularly gelatin.

Silicate microparticles for use in the injectable shear-thinning compositions of the present disclosure include natural silicate microparticles and synthetic silicate microparticles. Particular examples of silicate microparticles include natural and synthetic silicate layered clays. Natural silicate layered clays include montmorillonite, saponite, hectorite, kaolinite, palygorskite and sepiolite, among others. Synthetic silicate layered clays include lithium magnesium sodium silicates such as Laponite®-based silicate nanoplatelets (e.g., Laponite® XLG-based silicate nanoplatelets, Laponite® XLS-based silicate nanoplatelets, Laponite® XL21-based silicate nanoplatelets, and Laponite® D-based silicate nanoplatelets), Sumecton® SWN and Lucentite™ SWN, magnesium aluminum silicates such as Sumecton® SA, sodium magnesium silicates such as Optigel® SH and SUPLITE-MIP, and fluoromica such as Somasif™ ME100, among others.

Silicate microparticles for use in the injectable shear-thinning compositions of the present disclosure may have a size ranging from 5 nm or less to 75 nm or more in longest dimension (e.g., diameter for a sphere, length for a rod, greatest width for a plate-shaped particle, etc.), for example ranging anywhere from 5 nm to 10 nm to 25 nm to 50 nm to 75 nm in longest dimension.

The silicate microparticles for use in the injectable shear-thinning compositions of the present disclosure include microparticles that have a neutral charge, microparticles that have a net positive charge, and microparticles that have a net negative charge. The net charge of the silicate microparticles may depend upon the pH of the injectable shear-thinning composition. In some embodiments, the silicate microparticles have a net positive charge at the pH of the injectable shear-thinning composition. In some embodiments, the silicate microparticles have a negative charge at the pH of the of the injectable shear-thinning composition.

In some embodiments, the silicate microparticles are plate-shaped. In some embodiments, the silicate microparticles are silicate layered clays characterized by a discotic charge distribution on the surface. In some embodiments, the plate-shaped silicate microparticles comprise a positively charged edge and a negatively charged surface. In sone embodiments, the overall charge of the silicate microparticles is negative. In some embodiments, the plate-shaped silicate microparticles are from about 5 nm to about 60 nm in diameter, for example, from about 10 nm to about 40 nm in diameter, from about 10 nm to about 30 nm in diameter, or from about 20 to about 30 nm in diameter. In some embodiments, the plate-shaped silicate microparticles are from about 0.5 nm to about 2 nm in thickness, or about 1 nm in thickness.

In some embodiments, the injectable shear-thinning compositions of the present disclosure contain between 1 wt % and 15 wt % of one or more types of silicate microparticles, and typically contain from 1.5 wt % and 7.0 wt % (e.g., ranging anywhere from 1.5 wt % to 2.0 wt % to 2.5 wt % to 3.0 wt % to 4.0 wt % to 5.0 wt % to 6.0 wt % to 7.0 wt %) of one or more types of silicate microparticles, more particularly, lithium magnesium sodium silicate microparticles.

Block copolymers for use in the present disclosure include polyalkylene oxide block copolymers, polyalkylene oxide-polycaprolactone block copolymers, and polyalkylene oxide-polylactic acid block copolymers. Polyalkylene oxide block copolymers include those having two or more polyalkylene oxide blocks of differing chemical composition, for instance, two or more poly(C₁-C₆-alkylene oxide) blocks, including polyethylene oxide (PEO) blocks, polypropylene oxide (PPO) blocks, polybutylene oxide (PBO) blocks, etc., of differing composition. More particular examples of block copolymers include diblock copolymers (e.g., PEO-PPO diblock copolymers, PEO-PBO diblock copolymers, etc.), triblock copolymers (e.g., PEO-PPO-PEO triblock copolymers, PPO-PEO-PPO triblock copolymers, PEO-PBO-PEO triblock copolymers, PBO-PEO-PBO triblock copolymers, etc.), star block copolymers, which have a core region and three or more block copolymer arms coupled to the core region (for instance, R(PEO-PPO)ₙ, R(PPO-PEO)ₙ, R(PEO-PBO)ₙ, or R(PBO-PEO)ₙ, where R represents the core region and n is an integer of three or more, e.g., 3, 4, 5, 6, 7, 8, 9, 10 or more), and

In particular embodiments, the block copolymer is a PEO-PPO-PEO triblock copolymer having the formula,

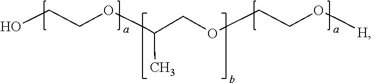

where a is an integer representing the PEO block lengths, and b is an integer representing the PPO block length. In some embodiments, a ranges from 10 to 500 or more, for example, ranging anywhere from 10 to 25 to 50 to 100 to 250 to 500, and b ranges from 10 to 300 or more, for example, ranging anywhere from 10 to 25 to 50 to 100 to 300. In one particular embodiment, the block copolymer is Pluronic® F-127, also known as poloxamer 407 which has approximate block lengths of a=101 and b=56. Typical molecular weight ranges from 10,000-14,600 g/mol. Commercial Pluronic® F127 samples may also contain ˜100 ppm butylated hydroxytoluene (BHT) as an antioxidant.

In some embodiments, the injectable shear-thinning compositions of the present disclosure contain between 0.05 wt % and 0.5 wt % of one or more types of block copolymer (e.g., ranging anywhere from 0.05 wt % to 0.075 wt % to 0.1 wt % to 0.125 wt % to 0.15 wt % to 0.175 wt % to 0.20 wt % to 0.225 wt % to 0.25 wt % to 0.30 wt % to 0.40 wt % to 0.50 wt %), more particularly, PEO-PPO-PEO triblock copolymers.

Radiopaque additives for use in the injectable shear-thinning compositions of the present disclosure include radiopaque microparticles that contain radiopaque metals and/or radiopaque metal compounds. Such radiopaque microparticles may be spherical or non-spherical. For example, the radiopaque microparticles may contain a radiopaque metal or metal compound throughout, may contain a cladding of a radiopaque metal or metal compound that surrounds a core of another material such as stainless steel, or may contain a core of a radiopaque metal or metal compound surrounded by a cladding of another material such as stainless steel. Radiopaque metals include transition metals such as tantalum, gold, platinum, tungsten, and alloys containing one or more of the same. Radiopaque metal compounds include compounds of barium, tantalum, bismuth, or gold such as barium sulfate, tantalum oxide, bismuth oxide, bismuth subcarbonate, bismuth oxychloride, gold oxide, tungsten oxide, and tungsten carbide.

Radiopaque microparticles for use in the injectable shear-thinning compositions of the present disclosure may have a size ranging from 5 nm to 70 microns in longest dimension (e.g., diameter for a sphere, length for a rod, greatest width for a plate-shaped particle, etc.), for example, ranging anywhere from 5 nm to 10 nm to 25 nm to 50 to 100 nm to 250 nm to 500 nm to 1 micron to 2.5 microns to 5 microns to 10 microns to 50 microns to 70 microns.

In some embodiments, the injectable shear-thinning compositions of the present disclosure contain between 10 wt % and 50 wt % of one or more radiopaque additives, and typically contain between 15 wt % and 40 wt % (e.g., ranging anywhere from 15 wt % to 20 wt % to 25 wt % to 30 wt % to 35 wt % to 40 wt %) of one or more types of radiopaque additives, typically tantalum microparticles.

In various embodiments, the injectable shear-thinning compositions in accordance with the present disclosure have a radiopacity that is greater than 100 Hounsfield units (HU), beneficially ranging anywhere from 100 HU to 250 HU to 500 HU to 750 HU to 1000 HU or more, for example, when measured on bench-top micro-CT systems such as Xtreme CT from Scanco Medical (Wangen-Brittisellen, Switzerland) or similar.

In various embodiments, the injectable shear-thinning compositions in accordance with the present disclosure have a radiopacity ranging from 30% to 93% contrast equivalent as measured by Fluoroscopy, or microCT

The water in the injectable shear-thinning compositions of the present disclosure may be provided in the form of ultrapure water, water for injection, saline, phosphate buffered saline, or high-ion-content water.

In some embodiments, the injectable shear-thinning compositions of the present disclosure contain between 50 wt % and 90 wt % of water, typically between 65 wt % and 85 wt % water (e.g., ranging from 65 wt % to 70 wt % to 75 wt % to 80 wt % to 85 wt % water).

The injectable shear-thinning compositions of the present disclosure may be formed using a variety of methods. The one or more types of fibrous proteins, one or more types of silicate microparticles, one or more types of radiopaque additives, one or more types of block copolymers, and water may be mixed in any suitable order. Mixing may be performed by any suitable mixing apparatus, including, for example, high shear mixers (e.g., Ross high shear mixer, Charles Ross & Son Company, Hauppauge, NY, USA), dual asymmetric centrifugal mixers (e.g., Hauschild SpeedMixer®, Hauschild SpeedMixer Inc., Farmington Hills, MI, USA), acoustic mixers (e.g., from Resodyn Acoustic Mixers, Inc., Butte, MT, USA), planetary centrifugal mixers (e.g., available from THINKY U.S.A., Inc., Laguna Hills, CA, USA), or speedmixers (e.g., FlackTek SpeedMixer® available from FlackTek, Landrum, SC, USA).

The injectable shear-thinning compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

In various embodiments, the injectable shear-thinning compositions of the present disclosure may contain one or more agents in addition to one or more types of fibrous proteins, the one or more types of silicate microparticles, one or more types of radiopaque additives, one or more types of block copolymers, and water. Examples of such additional agents include therapeutic agents, imaging agents, colorants, tonicity (osmolarity) adjusting agents, suspension agents, wetting agents, and pH adjusting agents.

Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, and STING (stimulator of interferon genes) agonists, among others.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the injectable shear-thinning compositions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, and (e) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others.

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity (osmolarity) adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes.

The injectable shear-thinning compositions of the present disclosure may be stored and transported in a sterile form. The injectable shear-thinning compositions may be shipped, for example, in a syringe, catheter, vial, ampoule, or other container.

In various embodiments, kits are provided, which may include one or more containers of shear-thinning compositions as described herein as well other components. For example, the kits may include one or more delivery devices for delivering the injectable shear-thinning compositions to a subject such as syringes, catheters or tubing sets. In some embodiments, the kits may comprise a shear-thinning composition as described herein preloaded in a catheter and/or a syringe barrel and/or in a container such as a vial or ampule. Alternatively or in addition, kits may be provided that include one or more accessory devices such as guidewires. Alternatively or in addition, the kits may be provided that include one or more containers of liquid materials (e.g. contrast agent, sterile water for injection, physiological saline, phosphate buffer, etc.). Alternatively or in addition, the kits may further comprise an additional therapeutic agent, which may be selected, for example, from those described above, among others. Instructions, either as inserts or as labels, indicating quantities of the composition to be administered and/or guidelines for administration can also be included in the kits provided herein. In some embodiments, the instructions comprise instructions for performing one or more of the methods provided herein.

The injectable shear-thinning compositions described herein can be administered by a variety of routes, depending upon the desired medical outcome. In some embodiments, the administering comprises injecting the injectable shear-thinning composition. In some embodiments, the injectable shear-thinning compositions are administered by parenteral administration. Parenteral administration includes intravenous, intraarterial, subcutaneous, intraperitoneal, or intramuscular injection or infusion. In some embodiments, the administering comprises an image guided procedure where computed tomography, fluoroscopy or ultrasound imaging is used to deliver the composition. In some embodiments, the administering comprises injecting the injectable shear-thinning composition into the vascular system of a subject. In some embodiments, the administering comprises injecting the injectable shear-thinning composition into a cancer of the subject or the vasculature supplying a cancer of the subject. In some embodiments, the administering is performed using a catheter and/or a syringe.

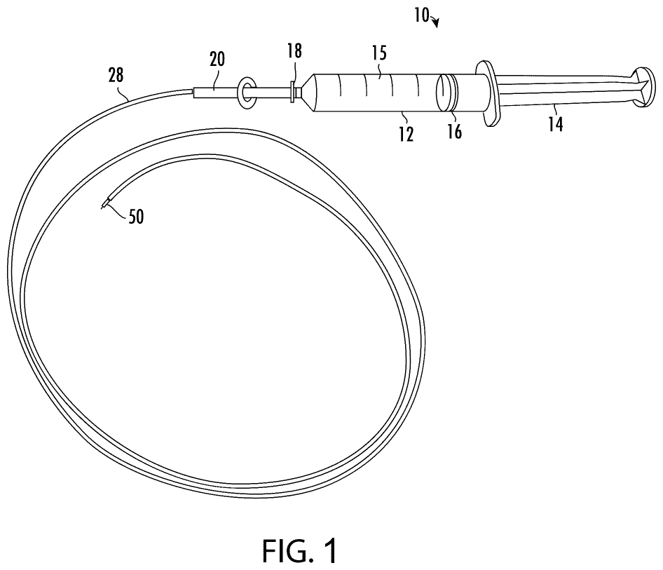

FIG. 1 illustrates an exemplary syringe 10 providing a reservoir for a shear-thinning composition as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a shear-thinning composition 15 for injection through the needle 50.

The injectable shear-thinning compositions described herein can be administered to patients for achieving a number of medical outcomes.

The injectable shear-thinning compositions described herein can be visualized (e.g., within a mammal) using any appropriate method during and/or after administration. For example, imaging techniques such as X-ray based imaging techniques including computerized tomography or X-ray fluoroscopy, magnetic resonance imaging and/or ultrasound can be used to visualize the injectable shear-thinning compositions provided herein.

The injectable shear-thinning compositions can be injected to provide spacing between tissues, the injectable shear-thinning compositions can be injected (e.g., in the form of blebs) to provide fiducial markers, the injectable shear-thinning compositions can be injected for tissue augmentation or regeneration, the injectable shear-thinning compositions can be injected as a filler or replacement for soft tissue, the injectable shear-thinning compositions can be injected to provide mechanical support for compromised tissue, the injectable shear-thinning compositions can be injected as a scaffold, the injectable compositions can be injected as an embolic composition, the injectable compositions can be injected as lifting agents for internal cyst removal, and/or the injectable shear-thinning compositions can be injected as a carrier of therapeutic agents in the treatment of diseases and cancers and the repair and regeneration of tissue, among other uses. The injectable compositions can also be injected into a left atrial appendage during a left atrial appendage closure procedure. In some embodiments, the injectable compositions may be injected into the left atrial appendage after the introduction of a closure device such as the Watchman® left atrial appendage closure device available from Boston Scientific Corporation.

The injectable shear-thinning compositions of the present disclosure may be used in a variety of medical procedures, including the following, among others: a procedure to implant a fiducial marker comprising the injectable shear-thinning compositions, a procedure to implant a tissue regeneration scaffold comprising the injectable shear-thinning compositions, a procedure to implant a tissue support comprising the injectable shear-thinning compositions, a procedure to implant a tissue bulking agent comprising the injectable shear-thinning compositions, a procedure to implant a therapeutic-agent-containing depot comprising the injectable shear-thinning compositions, a tissue augmentation procedure comprising implanting the injectable shear-thinning compositions, a procedure to embolize tissue, including benign tumors, malignant tumors and other abnormal tissue, a procedure to control bleeding, a procedure to introduce the injectable shear-thinning compositions between a first tissue and a second tissue to space the first tissue from the second tissue.

The injectable shear-thinning compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable shear-thinning compositions may be injected for the permanent or temporary occlusion of blood vessels, and thus may be useful for managing various diseases and conditions. For example, the injectable shear-thinning compositions may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the particulate composition.

Shear-thinning compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus, dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the particulate composition.

Shear-thinning compositions in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the particulate composition.

### Example 1

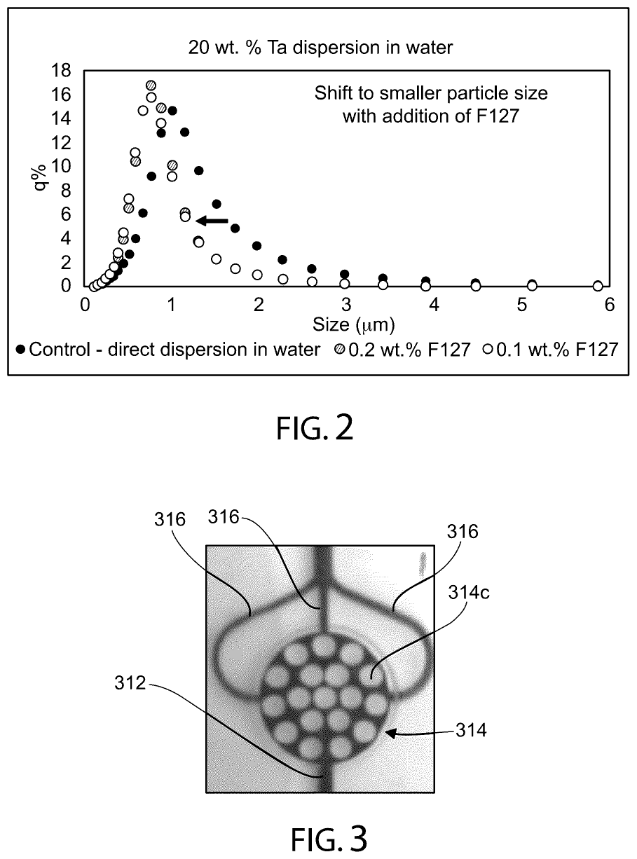

The following samples were made by dispersing 20 wt % tantalum microparticles in water alone or in water with 0.1 wt % or 0.2 wt % Pluronic® F127 (Sigma Aldrich, Milwaukee, WI, USA). Particle size distribution was measured by a Horiba LA960 laser scattering particle size distribution analyzer (Horiba, Ltd., Kyoto, Japan). The results are shown in the following Table and in FIG. 2. These results show a shift to smaller particle size with up to 0.2 wt. % Pluronic® F127 addition.

TABLE 1

| | Average diameter |
|---|---|
| Sample | (μm) |
| Tantalum in water only | 1.09 |
| Tantalum in water with | 0.77 |
| 0.2 wt % Pluronic ® F127 | |
| Tantalum in water with | 0.77 |
| 0.1 wt % Pluronic ® F127 | |

### Example 2

Samples were made from tantalum microparticles, Laponite® XLG (BYK-Chemie GmbH, Wesel, Germany), Type-A porcine skin gelatin (Sigma Aldrich, Milwaukee, WI, USA), 10 HGP 6500 porcine skin gelatin (Rousselot Inc., West Allis, WI, USA), Pluronic® F127 (one sample), and water in the amounts shown in Table 2.

TABLE 2

| | | | Type-A | | | |
|---|---|---|---|---|---|---|
| | | | porcine | 10 HGP | Pluronic ® | |
| | Tantalum | Laponite | gelatin | 6500 | F127 | |
| Sample | (wt %) | (wt %) | (wt %) | (wt %) | (wt %) | Water |
| 4NC85_30% | 30 | 2.00 | 0.248 | 0.106 | 0.0 | balance |
| 6500_30% Ta | | | | | | |
| 4NC85_20% | 30 | 2.00 | 0.283 | 0.071 | 0.0 | balance |
| 6500_30% Ta | | | | | | |
| 4NC85_20% | 30 | 2.00 | 0.283 | 0.071 | 0.2 | balance |
| 6500_30% | | | | | | |
| Ta_0.2% F127 | | | | | | |
| 4.5NC85_10% | 30 | 2.25 | 0.36 | 0.04 | 0.0 | balance |
| 6500_30% Ta | | | | | | |

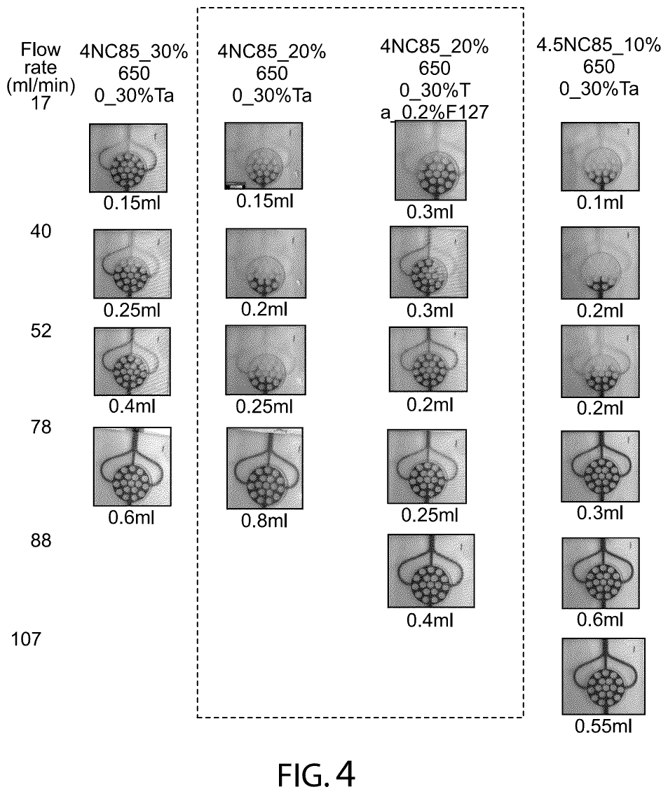

Samples were tested in an arteriovenous malformation (AVM) model like that described in Vollherbst D F, et al. Liquid Embolic Agents for Endovascular Embolization: Evaluation of an Established (Onyx) and a Novel (PHIL) Embolic Agent in an In Vitro AVM Model. AJNR Am J Neuroradiol. 2017 July; 38(7):1377-1382. With reference now to FIG. 3, the AVM model is made of silicone and includes an inlet tube 312, representing a feeding artery, a round, flat, honeycomb-like 3-dimensional structure 314 having a plurality of spaced-apart columns 314, and three outlet tubes 316, representing 3 draining veins, which is clamped between two glass plates. In FIG. 3, the fluid in the apparatus is black in color.

During testing, phosphate buffered saline was pumped at different rates into the inlet tube of the system with a constant-flow pump. A microcatheter was inserted to the entrance of the 3-dimensional space via a hemostatic Y-adapter and each of the formulations of Table 2, which are black in color due to the tantalum, is injected while at the same time flowing saline through the model at various flow rates.

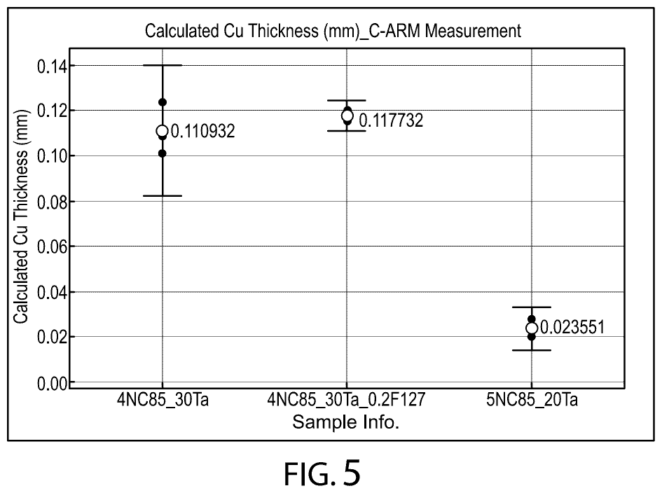

The results of these injections are shown in FIG. 4, where it can be seen that, at a low flow rate of 17 ml/min, the formulation with the Pluronic® F127 (4NC85_20%6500_30%Ta_0.2% F127) is able to penetrate further distal into the model, filling a larger area of the model relative to the comparable formulation without the Pluronic® F127 (4NC85_20%6500_30% Ta). At the same time the formulation with the Pluronic® F127 (4NC85_20%6500_30%Ta_0.2% F127) is able to withstand a higher flow rate (88 ml/min) during embolization of the model. The comparable formulation without the Pluronic® F127 (4NC85_20%6500_30%Ta) was not able to do so.

### Example 3

Samples were prepared as shown in Table 3.

TABLE 3

| | | | Type-A | | | |
|---|---|---|---|---|---|---|
| | | | porcine | 10 HGP | Pluronic ® | |
| | Tantalum | Laponite | gelatin | 6500 | F127 | |
| | (wt %) | (wt %) | (wt %) | (wt %) | (wt %) | Water |
| 4NC85_20% | 30 | 2.00 | 0.283 | 0.071 | 0.0 | balance |
| 6500_30Ta | | | | | | |
| 4NC85_20% | 30 | 2.00 | 0.283 | 0.071 | 0.2 | balance |
| 6500_30Ta_{—} | | | | | | |
| 0.2F127 | | | | | | |
| 5NC85_20Ta | 20 | 2.70 | 0.48 | 0 | 0.0 | balance |

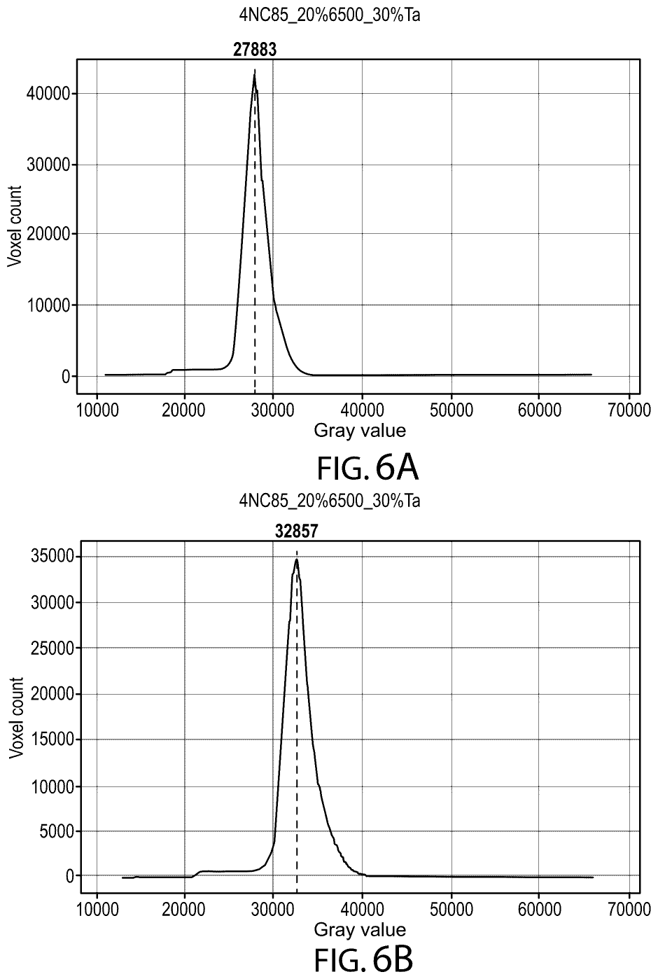

Equivalent copper thickness was calculated for each of the samples by calibration in imageJ with a benchmark of the copper step model and the results plotted in FIG. 5. Formulations are evaluated in terms of equivalence to a % contrast (Visipaque® 320, GE HealthCare, Chicago, IL, USA) rather than copper thickness in general. Interestingly, a 50% increase in tantalum resulted in an approximately 500% increase in calculated copper thickness.

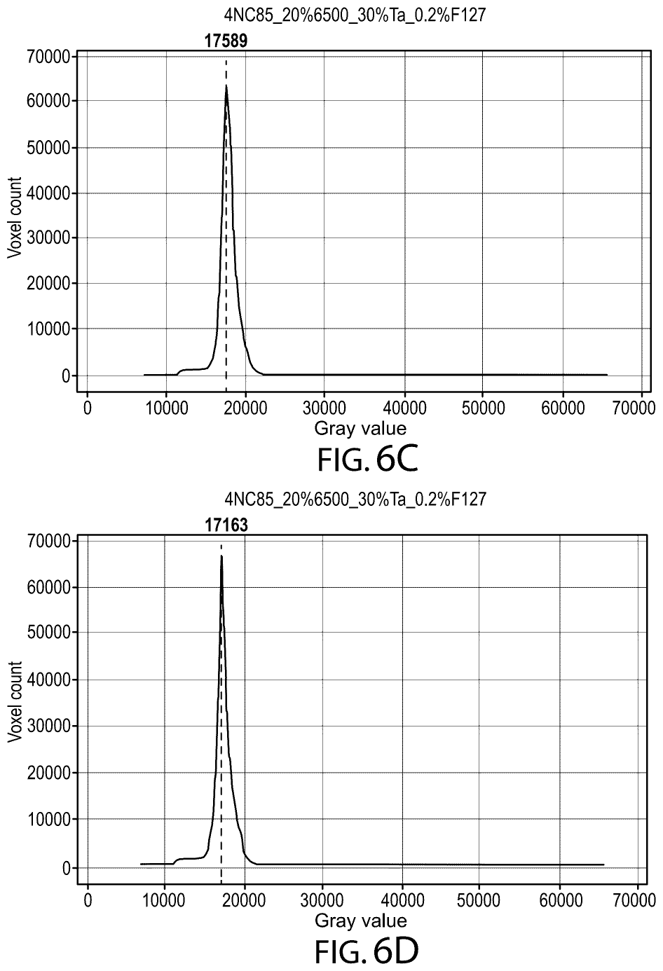

Micro-Computed Tomography (Micro-CT) analysis was performed on 4NC85_20%6500_30% Ta and 4NC85_20%6500_30%Ta_0.2%F127. Gray values at peak Voxel counts are plotted in FIGS. 6A-6D to compare radiopacity. Lower gray values indicate more radiopacity. The 4NC85_20%6500_30%Ta_0.2%F127 is 74% lower in peak voxel gray value compared to 4NC85_20%6500_30%Ta.

Although various embodiments are specifically illustrated and described herein, it will be appreciated that modifications and variations of the present disclosure are covered by the above teachings and are within the purview of any appended claims without departing from the spirit and intended scope of the present disclosure.

What is claimed is:

1. An injectable shear-thinning composition comprising (a) one or more types of fibrous proteins, (b) one or more types of silicate microparticles, (c) one or more types of radiopaque additives, (d) one or more types of block copolymers, and (e) water.

2. The injectable shear-thinning composition of claim 1, wherein the one or more types of fibrous proteins are collagen-based proteins.

3. The injectable shear-thinning composition of claim 2, wherein the one or more types of fibrous proteins comprise gelatin.

4. The injectable shear-thinning composition of claim 1, wherein the one or more types of silicate microparticles comprise natural and/or synthetic silicate layered clays.

5. The injectable shear-thinning composition of claim 1, wherein the one or more types of radiopaque additives comprise radiopaque microparticles that contain radiopaque metals or radiopaque metal compounds.

6. The injectable shear-thinning composition of claim 5, wherein the radiopaque microparticles are tantalum microparticles.

7. The injectable shear-thinning composition of claim 1, wherein the one or more types of block copolymers comprise a polyalkylene oxide block copolymer.

8. The injectable shear-thinning composition of claim 7, wherein the polyalkylene oxide block copolymer comprises one or more polyethylene oxide blocks and one or more polypropylene oxide blocks.

9. The injectable shear-thinning composition of claim 7, wherein the polyalkylene oxide block copolymer is a triblock copolymer that comprises two polyethylene oxide blocks and a central polypropylene oxide block between the polyethylene oxide blocks.

10. The injectable shear-thinning composition of claim 9, wherein the one or more polyethylene oxide blocks having a length between 10 and 500 monomer units and one or more polypropylene oxide blocks having a length between 10 and 300 monomer units.

11. The injectable shear-thinning composition of claim 1, wherein the injectable shear-thinning composition further comprises one or more additional agents selected from therapeutic agents, imaging agents, colorants, tonicity adjusting agents, suspension agents, wetting agents, and pH adjusting agents.

12. The injectable shear-thinning composition of claim 1, wherein the injectable shear-thinning composition is a sterile composition.

13. The injectable shear-thinning composition of claim 1, wherein the injectable shear-thinning composition is provided in a preloaded syringe.

14. A kit comprising a delivery device and one or more containers that contain an injectable shear-thinning composition that comprises (a) one or more types of fibrous proteins, (b) one or more types of silicate microparticles, (c) one or more types of radiopaque additives, (d) one or more types of block copolymers, and (e) water.

15. The kit of claim 14, wherein the delivery device comprises a syringe, a needle and optionally, a catheter.

16. A medical procedure comprising administering to a subject an injectable shear-thinning composition that comprises (a) one or more types of fibrous proteins, (b) one or more types of silicate microparticles, (c) one or more types of radiopaque additives, (d) one or more types of block copolymers, and (e) water.

17. The medical procedure of claim 16, wherein the method comprises injecting the injectable shear-thinning composition into the subject.

18. The medical procedure of claim 16, wherein the administering comprises parenteral administration.

19. The medical procedure of claim 16, wherein the administering is performed using a catheter or a syringe.

20. The medical procedure of claim 16, wherein the administering is performed under image guidance.
